(19) United States
(12) Patent Application Publication
(10) Pub. No.: US 2011/0281241 A1
(43) Pub. Date: Nov. 17, 2011

(54) ALUMINOUS CEMENT-BASED COMPOSITION FOR APPLICATION IN ENDODONTICS AND CEMENTITIOUS PRODUCT OBTAINED THEREOF

(75) Inventors: Victor Carlos Pandolfelli, Sao Carlos (BR); Ivone Regina de Oliveira, Araraquara (BR); Marcos Jacobovitz, Sao Carlos (BR); Hebert Luis Rossetto, Sao Carlos (BR)

(73) Assignee: FUNDACAO UNIVERSIDADE FEDERAL DE SAO CARLOS, San Carlos (BR)

(21) Appl. No.: 12/744,924

(22) PCT Filed: Nov. 27, 2008
(86) PCT No.: PCT/BR2008/000365

§ 371 Date: Oct. 27, 2010

(30) Foreign Application Priority Data
Nov. 27, 2007 (BR) PI0704502-6

(51) Int. Cl. A61C 5/02; A61C 5/04; A61K 6/06

(52) U.S. Cl. 433/224; 106/35; 523/117; 433/228.1

(57) ABSTRACT

The present invention provides a composition based on calcium aluminate cement (CAC) for application in endodontics, comprising: (a) a cement—Al₂O₃ (>68.5 wt %), CaO (<31 wt %), SiO₂ (0.3-0.8 wt %), MgO (0.4-0.5 wt %), and Fe₂O₃ (<0.3 wt %); (b) additives: dispersant at a content of 0.4 to 0.8% by weight of the cement, a plasticizer at a content of 2.0 to 4.0% by weight of the cement, and a radiopaque agent at a content of 20 to 35% by weight of the cement; and (c) water, wherein water/cement ratio lies in the range of 0.19-0.24 in the presence of additives. Cementitious product obtained thereof, after setting time, is also disclosed and characterized by enhanced properties when compared to the most used commercial repair cement, MTA.

### FIELD OF THE INVENTION

The present invention relates to the field of ceramic cements of hydraulic setting for using in dentistry, more specifically, to the composition based on calcium aluminate cement (CAC) for application in endodontics and to the cementitious product obtained thereof.

### BACKGROUND OF THE INVENTION

Endodontics is the specialty of dentistry which deals with the internal portion of teeth: coronal chamber (A), root canal (B), and periapical region (C), according to FIG. 1. Many clinical cases occurring from endodontic pathological alterations, whether traumatic or iatrogenic, are hardly prone to be treated by conventional therapies and may give rise to teeth extraction. It impacts not only the aesthetics, with psychological implications, but also the physiology of the stomatognathic system.

Fortunately, the aforementioned situations are pushing the search for materials and procedures to avoid such a radical intervention, what, on the other hand, leads endodontics to the multidisciplinary era. The premise of developing an “endodontic repair material” focused, at first, in matching its physical-chemical properties to appropriated physiological conditions for adjacent tissues. In other words, it means that such material should be non-carcinogenic, non-toxic, and insoluble in tissual fluids, in addition to dimensionally stable.

Examples of conventional repair materials, used in endodontic treatment, are: gutta-percha; resinous cements; cementitious compositions of zinc oxide and eugenol; and calcium hydroxide cements. Nevertheless, these materials do not have the ability to repair feasible pathological or iatrogenic defects of the endodontic structures, such as root perforations.

U.S. Pat. Nos. 4,652,593, 4,647,600 and 4,689,080 claim calcium hydroxide as addition to CAC cements in order to develop bactericide function.

U.S. Pat. Nos. 4,647,600 and 4,652,593 both disclose dental cements consisting of a composition A and a composition B, said composition A comprising (a) 100 parts by weight of a powder containing 20 to 70% by weight of calcium oxide and 30 to less than 80% by weight of aluminum oxide and treated on the surface with a selected inorganic/organic acid, and (b) 2 to 70 parts by weight of calcium hydroxide powder, and said composition B comprising of an aqueous solution containing 0.01 to 70% by weight of water-soluble high-molecular substance.

U.S. Pat. No. 4,689,080 discloses a base material composition for dental treatment that comprises 100 parts by weight of alumina cement powder, 1-50 parts by weight of calcium type powder hardening retarder, and 1-20 parts by weight of hardening retarder capable of restraining the calcium ion dispersion. It is affirmed that such composition gets hardened by low alkalinity and that it is very low in tissue irritation.

U.S. Pat. Nos. 5,415,547 and 5,769,638 both relate to the cement called MTA (Mineral Trioxide Aggregate) used as a repair material.

The same applies for papers from M. Torabinejad et al., “Physical and chemical properties of a new root-end filling material,” Journal of Endodontics, vol. 21, pp. 349-53 (1995) and M. Torabinejad et al., “Tissue reaction to implanted root-end filling materials in the tibia and mandible of guinea pigs,” Journal of Endodontics, vol. 24, pp. 468-71 (1998); M. Torabinejad et al., “Histological assessment of mineral trioxide aggregate as a root-end filling in monkeys,” Journal of Endodontics, vol. 23, pp. 225-28 (1997); C. F. Bates et al., “Longitudinal sealing ability of mineral trioxide aggregate as a root-end filling material,” Journal of Endodontics, vol. 22, pp. 575-78 (1996); M. Torabinejad et al., “Investigation of mineral trioxide aggregate for root-end filling in dogs,” Journal of Endodontics, vol. 21, pp. 603-08 (1995); M. Torabinejad et al., “Cytotoxicity of four root end filling materials,” Journal of Endodontics, vol. 21, pp. 489-92 (1995); M. Torabinejad et al., “Dye leakage of four root end filling materials: effects of blood contamination,” Journal of Endodontics, vol. 20, pp. 159-63 (1994); and M. Torabinejad et al., “Sealing ability of a mineral trioxide aggregate when used as a root-end filling material,” Journal of Endodontics, vol. 19, pp. 591-95 (1993).

MTA is composed by small hydrophilic particles, either white or gray, which harden in an aqueous medium, and its major constituents are: calcium oxide, aluminum oxide, and iron oxide (among other oxides in minor quantities), silicon dioxide, calcium sulfate, and bismuth oxide, the latter used to improve radiopacity of the product. In fact, it makes MTA more radiopaque than the other conventional materials, even more than dentin itself, being easily seen on radiographs.

Yet, the MTA breakthrough, compared to the others, derives from its biocompatibility to the dental and periodontal (lateral and apical) tissues. It is due to its capacity to dissociate Ca²⁺ and OH⁻ ions as soon as it is put in contact with water. Then, the initial pH of 10.2 rises to 12.5 after three hours of its mixture with water, a period wherein MTA setting took place. Because of such a high alkalinity, close to that of a calcium hydroxide solution, MTA is effective in turning its surrounding medium unpleasant for bacteria, as well as in inducing the formation of a hard tissue when used as repair material.

Indeed, the calcium hydroxide precipitation necrotizes a thin surrounding tissue, so that the carbon dioxide thus generated reacts with calcium hydroxide to form calcite crystals (calcium carbonate). At the same time, alkalinity itself drives the cells of conjunctive tissue to release a glycoprotein, the fibronectin, which favors the deposition of type-I collagen when in contact with the calcite crystals. As this process carries on, with the supplying of endogenous Ca²⁺, it causes the mineralization of such tissue, so-called “hard tissue”. All “in vivo” studies and clinical papers published so far have shown that MTA is not harmful to the surrounding tissues, either with none or low inflammatory responses.

MTA also presents other advantages when compared to other repair materials. For instance, it dismisses a dry area for application, provided the humidity activates its chemical hydration reaction. Indeed, most of the applications occur in a damp environment, what is not an inconvenience for MTA. Even when applied in human blood filled-cavities its potential of sealing was not damaged, which was experimentally verified by leakage tests between the material and the dental wall. Such sealing ability is also owed to a slight expansion that occurs during its hydration.

Actually, MTA hydration reaction leads to the formation of a colloidal gel which gets solid with an amorphous structure, responsible for mechanical compressive strength of 30 MPa after 24 h. After 21 days such mechanical strength reaches 40 MPa. It represents a relatively low strength when compared to that of amalgam, 312 MPa, what allows MTA to be used only as a “retro-obturator” or as a sealer of defects between the root and the lateral periodontium, because of the absence of direct loads in these cases.

This restricted utilization makes MTA expensive. That is why some researchers have tried Portland cement as an alternative to MTA. The results indicated that Portland cement has the same effects of MTA when applied in mice teeth pulps. However, the low purity of such material hinders its application in human beings. By the way, the material's cost itself does not reflect the product's final selling price, provided MTA composition is very close to that of neat Portland cement. Once again, the small volume of MTA consumption must bear all technical costs, what renders its final price.

In addition to the mechanical strength, other MTA weakness is the tooth darkening along the time, even when the whitish type is employed. The presence of iron compounds is the reason thereof. One additional disadvantage comes from the long setting time of MTA, in excess of 1 hour, because of the calcium sulfate in its composition.

U.S. Pat. No. 6,620,232 discloses a ceramic material for dental applications comprising a binder phase consisting of a cement-based system, of which at least 70 vol % consists of calcium aluminate cement in which the grain size is 10 μm or less, characterized in that the material comprises one or more additives adapted to give the material long term dimensional stability properties, said additive comprising Portland cement and/or some other organic Si-containing phase having a grain size of 0.5-10 μm and/or fine silica having a grain size of less 100 nm, at a content of 1-20 vol % of the material.

U.S. Pat. No. 6,969,424 discloses a method for the production of a chemically bound ceramic material by means of reaction between one or more powdered binding phase and a liquid, a quantity of powder containing said binding phase being suspended in said liquid so that all powder grains are brought into close contact with the liquid, whereupon the slurry thus obtained is drained so that the majority of surplus reacting liquid is removed, and is compacted during final draining, before the material is permitted to harden by the reaction between said binding phase and the remaining liquid. One or more expansion-compensating additives, adapted to give the material dimensionally stable long-term properties, are mixed into said powder, prior to or in conjunction with its suspension in the liquid. The same is valid for U.S. Patent Application Publication 20060167148A1.

Axen, U.S. Patent Application Publication 20040237847, discloses a chemically bonded ceramic material based on calcium aluminate hydrate with at least one inert additive, calcium titanate for instance, to make the material biocompatible for implants, particularly for dental and orthopedic applications.

Besides the technological developments in the field of this invention, there still is a need for a composition based on calcium aluminate cement (CAC), for using in endodontics, composed of Al₂O₃ (≧68.5 wt %), CaO (≦31 wt %), SiO₂ (0.3-0.8 wt %), MgO (0.4-0.5 wt %), and Fe₂O₃ (<0.3 wt %), with additives such as a polymeric polyglycol-based dispersant at a content of 0.4 to 0.8% by weight of the cement (preferentially 0.6 wt %), a plasticizer agent like CaCl₂ at a content of 2.0 to 4.0% by weight of the cement (preferentially 2.8 wt %), and a radiopaque agent like ZnO at a content of 20 to 35% by weight of the cement (preferentially 25 wt %), said composition comprising water at a water/cement ratio (w/c) in the range of 0.19 to 0.24, in the presence of additives, such composition and method to prepare it being described and claimed in this present application.

### SUMMARY OF THE INVENTION

In a general embodiment, this present invention discloses a composition based on calcium aluminate cement (CAC) for application in endodontics, comprising Al₂O_{3 (≧}68.5 wt %), CaO (≦31 wt %), SiO₂ (0.3-0.8 wt %), MgO (0.4-0.5 wt %), and Fe₂O₃ (<0.3 wt %), with additives such as a polymeric polyglycol-based dispersant at a content of 0.4 to 0.8% by weight of the cement, a plasticizer agent like, but not limited to, CaCl₂ at a content of 2.0 to 4.0% by weight of the cement, a radiopaque agent like ZnO at a content of 20 to 35% by weight of the cement, and water at a water/cement ratio (w/c) in the range of 0.19 to 0.24.

Thus, this invention provides an aluminous cement composition for application in endodontics based on calcium aluminate cement (CAC) with additives such as dispersant, plasticizer, and radiopaque agent.

This invention also provides an aluminous cement composition for application in endodontics wherein the setting time is controlled by the addition of citric acid or lithium carbonate.

The present invention provides an aluminous cement composition for application in endodontics so that improvements on rheology are obtained after the addition of dispersants, preferentially those of the polyglycol family, and plasticizers, preferentially CaCl₂, the first one being responsible for dispersing the materials' particles and thereby improving the workability, in addition to lowering the water consumption and rendering higher material density.

The invention herein provides an aluminous cement composition for application in endodontics wherein the mechanical strength improvement is obtained by the addition of the dispersants, preferentially those of the polyglycol family that reduce the w/c ratio and allow the formation of hydrates with higher capacity of filling the interparticle voids, which give the material a higher density and a lower porosity, as well as lower pore sizes.

At last, this invention provides an aluminous cement composition for application in endodontics wherein radiopacity is obtained by the addition of radiopaque agents, like ZnO, preferentially, which gives the material an adequate radiopacity without inducing toxicity, in addition to a higher mechanical strength and smaller porosity.

### BRIEF DESCRIPTION OF THE FIGURES

FIG. 1 schematically illustrates the anatomic sites of interest in the field of endodontics.

FIG. 2 is a graph which shows temperature in function of time for aqueous suspensions of calcium aluminate cement (curve 1) and gray-MTA (curve 2).

FIG. 3 is a graph which shows compressive strength as a function of the curing time for: samples of plain CAC (curve 1); CAC samples with additives, that is, dispersant and plasticizer (curve 2); CAC samples with additives plus radiopaque (curve 3); and samples of plain MTA, the most used endodontic repair cement (curve 4).

FIG. 4 is a graph which shows apparent porosity as a function of the curing time for: samples of plain CAC (curve 1); CAC samples with additives, that is, dispersant and plasticizer (curve 2); CAC samples with additives plus radiopaque (curve 3); and samples of plain MTA, the most used endodontic repair cement (curve 4).

FIG. 5 is a graph which shows pore size distribution evaluated by mercury porosimetry technique for: the samples of calcium aluminate cement containing additives plus radiopaque (curve 1); and the plain MTA samples, the most used endodontic repair cement (curve 2).

FIG. 6 is a graph which shows pH as a function of the time for water in contact with samples of plain CAC (curve 1) and MTA (curve 2), both at the end of setting time.

FIG. 7 is a graph which shows ionic conductivity as a function of the time for aqueous solutions in equilibrium with samples of plain CAC (curve 1) and MTA (curve 2), both at the end of setting time.

FIG. 8 schematically illustrates the splitting test of a dental material.

FIG. 9 schematically illustrates the “in vitro” test apparatus for bacterial infiltration.

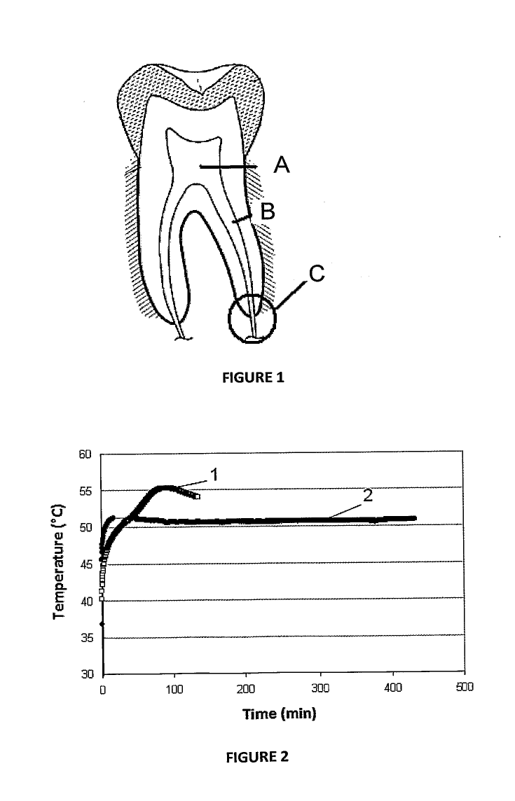

### DETAILED DESCRIPTION OF THE INVENTION

The present invention discloses an alternative material based on calcium aluminate cement which can be used as repair cement in endodontics. One of the main aspects of the invention is the relation between calcium aluminate cement and additives for applications in endodontics. Other aspect is related to the process to prepare such composition. Additionally, this invention also discloses the product yielded by the calcium aluminate cement hydration.

Calcium aluminate cements (CAC), sometimes also called aluminous or high-alumina cements, can be produced by fusing Al₂O₃ and CaCO₃ blend or through calcination process at temperature range of 1315 to 1425° C., the latter being the most viable method for production of CACs with uniform compositions. In the fusion method, Al₂O₃ and CaCO₃ are submitted to temperatures between 1450 and 1550° C. inside electric arc furnaces. The calcium aluminate formed is cooled and then crushed to the required particle size distribution.

In a general approach, the formation of CAC can be described through the following chemical reaction, Eq. 1:

CaCO₃+Al₂O₃→Ca(AlO₂)₂+CO₂↑ (1)

Despite the simplicity of the chemical reaction described above, the CAC formation occurs in a complex way. At the beginning of the blend fusion process, crystalline aluminate phases with high content of Ca are formed. With the increase of the temperature, more CaO and Al₂O₃ react with the products formed at the beginning, producing other phases with lower Ca content.

In this process, quantity and type of the formed calcium aluminate crystalline phase will both depend on the CaO and Al₂O₃ ratio in the mixture, on the temperature reached and on the cooling process of the material. Cooling interferes on the type of the crystalline phase, provided crystallization may occur on larger or lesser extent, depending on the temperature gradient inside the fused block.

Under the production condition currently used, high purity CAC phases are obtained with different proportions among the various possible crystalline phases, depending on the material applications. These phases show distinct properties regarding reactivity with water, mainly because, in general, the higher the quantity of CaO, the higher the reactivity.

Besides the diversity of possible phases, the commercial calcium aluminate cements are mainly constituted by three main phases which are responsible for the hydraulic setting process: the anhydrous phase CA (CaO.Al₂O₃), comprising about 40 to 70% of the product; CA₂ (CaO.2Al₂O₃), which is the second in proportion (<25%), and the C₁₂A₇ phase (12CaO.7Al₂O₃), at a content of 10% or less.

CAC binder does not present the weaknesses related to MTA, provided it has a white color (close to the dental structure appearance) and iron content at a trace level which inhibits teeth darkening, commonly seen when MTA is used as repair material. In addition, CAC has a very low content of free magnesium and calcium oxides what discourages unwanted delayed expansion in contact with water. Moreover, CAC is a hydraulic binder, like MTA, which reacts with water to obtain its mechanical strength. Thus, its hydration reaction is favored in humid environment like dental cavities.

Regardless the proportion of crystalline phases present in CAC, its hardening process starts after the reaction with water, resulting in calcium (Ca²⁺) and tetrahydroxi-aluminate (Al(OH)⁴⁻) ions dissolution. This process carries on until the solution reaches a saturation level sufficient to promote massive precipitation, followed by crystal growth of the phases known as calcium aluminate hydrates. This process tends to form strong connections among neighboring particles, rendering material's mechanical strength therefrom. The hydration kinetic and the precipitation of the formed hydrate are strongly dependent on the crystalline composition of the former material as well as the structure of the materials that form during the precipitation. The latter is predominantly influenced by the amount of available water, by the reaction time, by the temperature and by the concentration between ratio Ca²⁺ and Al(OH)⁴⁻ ions which are released in solution during the dissolution process of the anhydrous phases.

The development of the present calcium aluminate cement composition was carried out by the introduction of additives that improved its physical and biological properties. This composition presents features able to overcome MTA, as follow: (i) adequate rheology to placement into pulp cavity due to the choice of suitable dispersant additive, among others; (ii) reduced porosity with lower pores size as well, and, mainly, (iii) higher mechanical strength.

Hence, by the addition of a dispersant and a plasticizer it is possible to obtain calcium aluminate cement pastes with reduced w/c ratio when compared to MTA, what was seen by the rheological tests whose results have driven this patent application. Deionized water was used in the preparation of the compositions disclosed in this invention, suitable for dental products.

Dispersant agents include citric acid (and its salts like sodium citrate), polyacrylic or polymethacrylic acid (and its salts like sodium polymethacrylate), phosphoric acid (and its salts like sodium hexamethaphosphate), and the polyglycol family. In a preferred embodiment, the dispersants are those of the polyglycol family, for example, modified polycarboxylate ether. The plasticizers include CaO, CaCl₂, Ca(OH)₂, and CaCO₃; the preferred plasticizer is CaCl₂. The radiopaque agents include ZrO₂, ZnO, SnO₂, BaSO₄, Bi₂O₃, Bi₅O(OH)₉(NO₃)₄, and CHI₃. The preferred radiopaque agent is ZnO.

Calcium aluminate cement also presents a setting time significantly lower than that of MTA, shown by heat release tests. The increase of temperature, typical of hydration reaction, was not observed until 400 minutes to MTA suspension (curve 2 of FIG. 2) while heat releasing occurred in CAC suspensions (curve 1 of FIG. 2) approximately after 60 minutes of contact with water. Moreover, the setting time of CAC can be properly fitted to clinical needs by additives that act retarding (citric acid or its salts, including sodium citrate) or accelerating (lithium salts, including lithium carbonate) its hydration mechanism.

Thus, retarders are so-called by their action of lengthening the cements setting time. Generally, the retarding mechanism of the additives is attributed to an increase of the time required for the anhydrous phase dissolution to reach Ca²⁺ and Al(OH)⁴⁻ concentrations enough to trigger induction period (solution saturation). In fact, it is due to the strong retarder affinity to calcium cations, leading to formation of less soluble hydrates which are richer in Ca²⁺ ions and hinder faster precipitation.

Contrarily, accelerators act by changing the process of hydrate precipitation decreasing the time necessary to the nucleation. In other words, it allows the precipitation to occur as soon as the saturation is reached. Also, they have crystalline structure necessary to serve as germs for the nucleation of the hydrates.

Typical contents at which retarders are used are in the range of 0.02 to 0.5% by weight of CAC, whereas accelerators are used at content of 0.005 to 0.1% by weigh of CAC.

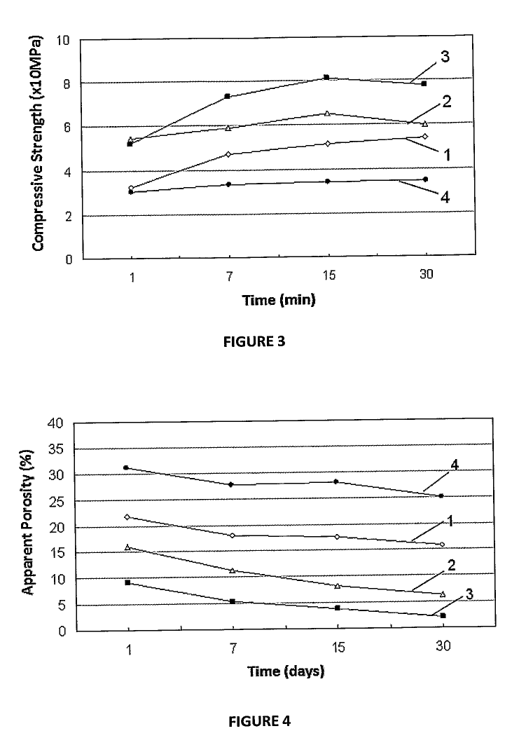

Another feature that indicates the advantages of the use of CAC to replace the MTA is the mechanical strength. CAC presented compressive strength of 30 MPa, within 24 hours of setting, which increased to 50 MPa after 15 days, against 30 MPa evaluated for the MTA. With the incorporation of additives, dispersant and plasticizer, to CAC, the compressive strength reached 65 MPa (after 15 days of curing). Moreover, the addition of radiopaque additive (ZnO, although not limited to it) helped to increase the compressive strength with the advantage of not inducing toxicity to material as Bi₂O₃, present in the MTA, does. Thereby, the compressive strength of 80 MPa for CAC paste was obtained while the MTA compressive strength remained below 40 MPa (FIG. 3).

In addition, the results for CAC apparent porosity also indicated a very low porosity when compared to the MTA, even lower when in presence of additives. After curing time of 15 days, the neat CAC porosity was lower than 21% while for MTA the porosity was of 31%. With the incorporation of additives (dispersant and plasticizer) to CAC, the porosity dropped to less than 10%. Addition of the radiopaque agent contributed to further reduce CAC porosity to 4% after curing time of 15 days (FIG. 4). In addition to the lower porosity, CAC presented pores with smaller diameter (0.25-0.55 μm) compared to that of MTA (2.5 μm), as measured by mercury porosimetry technique (FIG. 5).

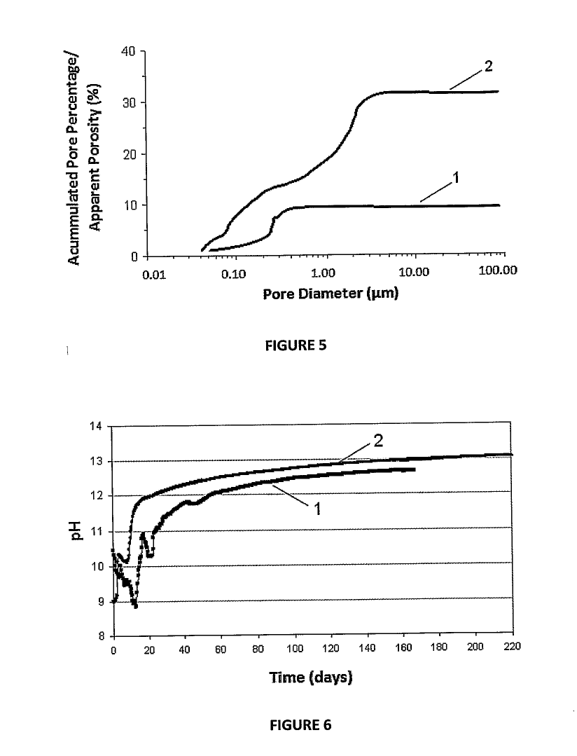

On the other hand, CAC has some similarities to the MTA. It is also non-carcinogenic, non-toxic and able to turn the medium where it is inserted highly alkaline (FIG. 6). Its ability to promote increase in the medium pH makes it biocompatible when compared to the other commercial materials. This alkalization derives from CAC hydration reaction, particularly due to dissociation of Ca²⁺ e OH⁻ ions when the material comes in contact with water, similar to MTA. It creates an unpleasant environment for bacteria to survive and, still, it can stimulate the formation of a mineralized tissue barrier when used as repair material. Antimicrobial activity is related to the release of hydroxyl ions while the formation of a barrier is promoted by the release of Ca²⁺ in adjacent tissues.

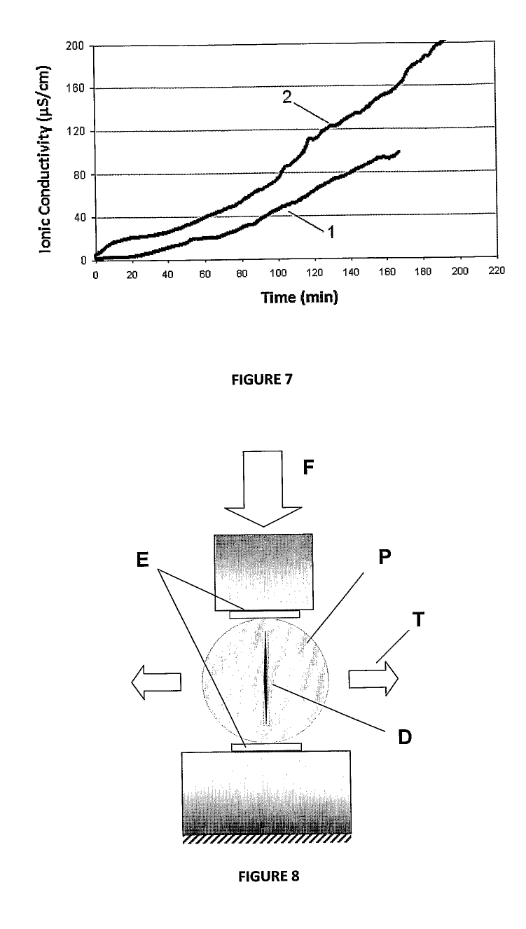

The release of calcium ions in the medium was confirmed by the increase of ionic conductivity in aqueous solution (FIG. 7). The pH and ionic conductivity tests also showed that CAC presents greater resistance to disintegration in an aqueous environment. On the other hand, MTA presented significant disintegration that may have contributed to overcome the conductivity measured for the CAC.

Preliminary biological tests pointed out encouraging results concerning the therapeutic use of CAC as an endodontic repair material: an incipient microbiological experiment allowed assessing, through “in vitro” model, the ability of CAC and MTA to block bacterial microleakage when used as root canal fillers. All specimens of the positive control group, the canals without cements, showed contamination, whereas the negative ones did not show any contamination. Both evaluated materials, CAC and MTA, showed absence of microbial growth, what gives them capacity to avoid bacterial microleakage when utilized as root canal fillers.

A second biological test about biocompatibility of CAC further validated its properties: an “in vitro” study aimed to assess the progress of an initial culture of osteogenic cells derived from newborn mice calvaria in contact with tiny discs of MTA and CAC. At 3 and 7 days, the total number of cells was significantly higher upon the sheets containing CAC when compared to that of MTA. Morphologically, these cells were all adhered and spread over the control glass sheets of both materials although an inhibition zone for cell growth was noticed only for MTA sample.

The results for in situ alkaline phosphatase activity, determined through histological Fast Red method, suggested that at the end of proliferative phase of culture, around 7 days, the presence of CAC favored the differentiation of a larger number of osteoblastic line cells with alkaline phosphatase activity when compared to MTA.

The materials employed in the present invention were all commercial products, such as the calcium aluminate cement. MTA (gray or white) was taken as reference and assessed as received, with no additives. The chemical composition of calcium aluminate cement comprises Al₂O_{3 (≧}68.5 wt %), CaO (≦31.0 wt %), SiO₂ (0.3-0.8 wt %), MgO (0.4-0.5 wt %) and Fe₂O₃ (<0.3 wt %). MTA is composed by Portland cement (75 wt %), bismuth oxide (20 wt %) and dihydrate calcium sulfate (5 wt %). Portland cement, in turn, is composed by SiO₂ (21.2 wt %), CaO (68.1 wt %), Al₂O₃ (4.7 wt %), MgO (0.48 wt %) and Fe₂O₃ (1.89 wt %).

The additives used were: (a) a polymeric dispersant of polyglycol family at a content of 0.4 to 0.8% by weight of cement, preferentially 0.6 wt %; (b) a plasticizer, CaCl₂, at a content of 2.0 to 4.0% by weight of cement, preferentially 2.8 wt %; and (c) a radiopaque agent, ZnO, at a content of 20 to 35% by weight of cement, preferentially 25 wt %.

To blend cement with additives, the procedure is as follow: composition is prepared by mixing all additives—dispersant, plasticizer, and radiopaque agent—to aluminous cement which, thereafter, is homogenized inside a flask with 40-80 g of Al₂O₃ mill balls per 100 g of cement plus additives. Homogenizing is performed in a double-cone mill through 1 to 3 h.

In another preferred embodiment, calcium aluminate cement setting time is adjusted by the addition of other additives, i.e. retarders or accelerators, such as citric acid at a typical content of 0.02 to 0.5% by weight of cement or lithium carbonate at a content of 0.005 to 0.1% by weight of cement, respectively.

Physical-chemical characterization of the materials was performed by rheological, pH, ionic conductivity, and heat release tests, in addition to porosity and mechanical strength measurements.

Biological evaluation enclosed “in vitro” histopathological and bacterial microleakage tests, characterizing material's biocompatibility by cell culture in contact therewith.

It is worth emphasizing that compressive strength of CAC may surpass 200 MPa after compaction of the cementitious paste which fills dental cavities. In other words, it opens great perspectives for applications of this material other than root canal filling, such as canal filling and pulp capping.

In addition to the high mechanical strength, CAC may overcome current dental repair materials because of: (i) its alkaline character, not harmful to dental pulp, what gives the material the ability to replace zinc phosphate with acid character; and (ii) its stickless behavior to dental instruments, contrarily to glass-ionomer cement.

CAC recommendation for such applications could be supported by its properties such as rheological ones, which match those of a supposed endodontic repair material and exceed with respect to plasticity, homogeneity, and workability, including proper setting time induced by specific additives.

It is important to emphasize that improving versatility could imply in enlarging CAC usability as endodontic cement. In turn, the enlarging of consumption triggers a higher volume of CAC production, and then, lower marginal costs, what could help to reduce the selling price. In other words, it would permit to more people to take the benefits of such therapy, provided the current treatment cost is too high, at least in emerging countries like Brazil.

Those skilled in the art will appreciate that the present invention can be implemented with other embodiments without changing the technical spirit or essential features thereof. Therefore, the above described embodiments, as well as the following examples, should be appreciated as having been disclosed for illustrative purposes and are not restrictive. Still, those skilled in the art will appreciate that the scope of the present invention is defined by the accompanying claims rather than the above detailed description, and various modifications, additions and substitutions, derived from the meaning and scope of the claims and equivalent concepts thereof, belong to the scope of the present invention.

### Example 1

Manipulation tests consisted of powder mixture of different types of material, one at a time, with water on a glass plate to determine visually the water-to-cement ratio (grams of water per gram of cement) necessary to obtain a homogeneous paste with ideal viscosity for placement. These tests were also conducted in the presence of various additives to verify their influence on the paste viscosity as well as on the water consumption. The water/cement ratio obtained for CAC was preferentially 0.32, although such ratio was reduced preferentially to 0.21 after additives incorporation.

### Example 2

The temperature tests consisted of measuring the heat released by cement paste as a function of time, which supply important information about reaction kinetic. The temperature evolution of hydraulic binder pastes can be registered with the help of a thermocouple and used as a measure of the hydration of cements. This sensor was inserted into the plain cement suspensions (water/cement=1.5) and the measurements were thereafter initiated. A capture system (ESA 9800, Matec Applied Sciences-UK) was connected to the thermocouple to record data automatically. Hydration kinetic of cement is thereby measured provided this reaction, responsible for cement hardening, is followed by temperature rising. Suspensions were kept at 50° C. during the heat evolution measurements, because it provides a faster hydration process than at room temperature. In this way, CAC presented a setting time significantly lower than that of MTA.

The following Table 1 compiles temperature data against time for both cements, CAC and MTA.

TABLE 1

TEMPERATURE (° C.)

| Time | CAC without | Gray MTA |
|---|---|---|
| (min) | additives | (control) |
| 0 | 42 | 45 |
| 40 | 52 | 51 |
| 80 | 56 | 51 |
| 120 | 53 | 51 |
| 160 | 51 | 51 |
| 200 | — | 51 |
| 300 | — | 51 |
| 400 | — | 51 |

### Example 3

The characterization of alkalinity was carried out by pH and ionic conductivity tests of the proposed endodontic materials. The pH measurements of aqueous solutions were taken at the end of setting time. At the same time, the ionic conductivity was measured and correlated to calcium ions dissociation.

Aqueous suspensions of the different types of cements (water/cement=0.35) were prepared and cast into 20 mm diameter×20 mm height cylinder molds. These samples were cured at 50° C. during 24 hours, dried at 110° C. for 72 hours and added to a container with water (80 mL). Sensors connected to an automatic data capture system (ESA 9800, Matec Applied Sciences-UK) were inserted into water and the pH and ionic conductivity measurements as a function of time were immediately initiated. Container was adapted to allow the temperature control during measurements at 30° C.

CAC presented anti-bacterial properties in function of such pH development, similar to that of MTA.

Table 2 below compiles data of pH measurement against time for both cements, CAC without additives and MTA.

TABLE 2

pH

| Time | CAC without | Gray MTA |
|---|---|---|
| (min) | additives | (control) |
| 0 | 9.00 | 9.02 |
| 20 | 10.20 | 11.96 |
| 40 | 11.79 | 12.29 |
| 60 | 12.07 | 12.51 |
| 80 | 12.31 | 12.64 |
| 100 | 12.45 | 12.74 |
| 120 | 12.54 | 12.84 |
| 140 | 12.61 | 12.91 |
| 160 | 12.63 | 12.96 |
| 180 | — | 13.00 |
| 200 | — | 13.03 |

CAC also presented biocompatibility in contact with cells and tissues owing to Ca²⁺ ions release, one of the most important constituents of dental tissues. This release was assessed by the rise in the ionic conductivity of cementitious medium up to the setting time.

Ionic conductivity data are compiled in Table 3, as follows below.

TABLE 3

Conductivity (μS/cm)

| Time | CAC without | Gray MTA |
|---|---|---|
| (min) | additives | (control) |
| 0 | 0 | 0 |
| 20 | 3.62 | 20.42 |
| 40 | 11.43 | 25.90 |
| 60 | 19.69 | 39.57 |
| 80 | 29.61 | 53.95 |
| 100 | 45.78 | 75.08 |
| 120 | 63.37 | 110.71 |
| 140 | 78.79 | 131.79 |
| 160 | 92.60 | 151.96 |
| 180 | — | 184.18 |

### Example 4

Mechanical strength and apparent porosity tests were carried out to characterize the endodontic materials. Cementitious pastes of the different cements were prepared either in the absence of additives (w/c=0.35) or in the presence of different additives (w/c=0.32) and cast into 20 mm diameter×25 mm height cylinder samples. A set of samples was cured at 50° C. in acclimatized chamber (Vötsch, model 20-20) in a moisture-saturated environment (≈100% RH) during 1, 7, 15 and 30 days. Humid samples (immediately after curing) were submitted to tensile strength tests as a function of curing time whereas those dried at 110° C. for 3 days were submitted to apparent porosity measurements.

Mechanical strength was measured according to the ASTM C496-90 standard, the so-called splitting tensile strength, using a universal test machine (MTS Systems Corp., mod. 810, USA), at a constant loading rate of 11 N/s, each set with at least 5 samples. The splitting tensile strength was calculated by Equation (2), where: σ_{R} is the rupture stress (MPa); P is the ultimate load (N); h (mm) and d (mm) are height and diameter of the samples, respectively.

$${σ}_{R} = 2 ( \frac{{P}_{max}}{π hd} ) \qquad ( 2 )$$

In this test, the samples are submitted to diametral compression forces performed by the equipment, generating tensile stress inside the sample. Crack (D) propagation occurs in the vertical direction provided that main tensile stresses (T) are perpendicularly developed to the compressive forces (F), crossing all the cylinder (P) extension (FIG. 8). Each sample is placed between two strips (E).

The apparent porosity was evaluated according to the immersion test (Archimedes principle), using kerosene as the immersion liquid. This principle consists of weighing sample in the dry condition (W_{d}), in the humid condition (Wₕ) after 1 h of immersion in the liquid under vacuum, and in the immersed condition (Wi), in the same liquid.

Thereby, the apparent porosity is calculated through Equation (3) below:

$$A . P . = ( \frac{( {W}_{h} - {W}_{d} )}{( {W}_{h} - {W}_{i} )} ) \times 100 \qquad ( 3 )$$

Also, the pore size distribution for both cements, CAC with additives and MTA, was evaluated by Mercury Intrusion Porosimetry (MPI). The underlying of this technique relates the pressure of mercury intrusion to the pore volume, and is based on the non-wetting behavior of mercury in contact with the intruding material. In other words, mercury cannot spontaneously penetrate the material unless a pressure is applied on it. The result, taken over a range of pressures, gives a curve with the pore volume versus pore size distribution for the sample.

CAC presents a significantly higher mechanical strength when blended with additives such as dispersants like citric acid, polyacrylates, and those of polyglycol family, for instance. It is attributed to particles dispersion which, ultimately, gives the material a well packed and less porous microstructure. The polyglycol family dispersants are more effective in yielding higher mechanical strength due to its superior performance and to the privilege to forming phases like C₃H₆ and AH₃. The latter is gel-like and occupies the interstitial voids, with consequences such as lesser porosity and higher mechanical strength. The CAC mechanical strength may be further improved with the addition of plasticizer and radiopaque agent to former composition, resulting in a huge increase in comparison to MTA mechanical strength.

Mechanical strength against time data are compiled in Table 4 for neat CAC, CAC with additives (dispersant and plasticizer), CAC with additives plus radiopaque agent, and MTA.

TABLE 4

COMPRESSIVE STRENGTH (MPa)

| | | | CAC + | |
|---|---|---|---|---|
| | Neat | CAC + | additives + | Gray MTA |
| Days | CAC | additives | radiopaque | (control) |
| 1 | 32.0 | 54.4 | 52.1 | 30.2 |
| 7 | 47.2 | 59.0 | 73.0 | 33.4 |
| 15 | 51.2 | 65.2 | 81.3 | 34.3 |
| 30 | 54.0 | 59.6 | 77.7 | 34.4 |

The improvement of the mechanical strength obtained with additives is followed by the reduction of material's porosity, resulting in a material with significantly smaller pore sizes when compared to MTA.

The porosity drop along the time, between 1 and 30 days, is shown in Table 5 below.

TABLE 5

APPARENT POROSITY (%)

| | | | CAC + | |
|---|---|---|---|---|
| | Neat | CAC + | additives + | Gray MTA |
| Days | CAC | additives | radiopaque | (control) |
| 1 | 21.74 | 16.03 | 9.17 | 31.19 |
| 7 | 17.96 | 11.33 | 5.40 | 27.70 |
| 15 | 17.61 | 8.12 | 3.78 | 28.02 |
| 30 | 15.89 | 6.31 | 2.03 | 25.15 |

Pore accumulated percentage to apparent porosity relation is compiled in Table 6 for CAC with additives plus radiopaque agent, in function of pore diameter, and also for MTA.

TABLE 6

Pore accumulated %/Apparent porosity

| Pore | CAC + | |
|---|---|---|
| Diameter | additives + | Gray MTA |
| (μm) | radiopaque | (control) |
| 0.1 | 1.83 | 8.37 |
| 1.0 | 10.0 | 18.83 |
| 10 | 9.17 | 32.00 |
| 100 | 9.17 | 32.00 |

### Example 5

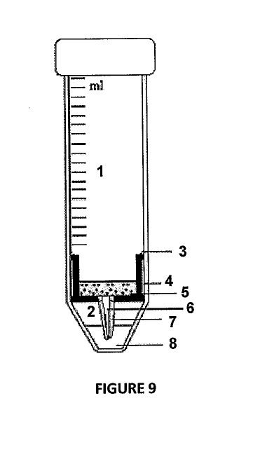

Bacterial microleakage tests were carried out in order to evaluate both endodontic cements, CAC and MTA, used as root canal fillers, through “in vitro” model. Falcon tubes were prepared for such evaluation, and to do so they were divided into two chambers separated by an apparatus containing a human tooth whose root canal links both chambers (FIG. 9). In FIG. 9, (1) is the superior chamber; (2) is the inferior chamber; (3) is the lateral sealed with silicone; (4) is a plastic resin tube; (5) is the microbial culture; (6) is the dental root; (7) is the root canal filler; and (8) is the transport fluid RTF. The internal portion (2) receives a fraction of the transport fluid RTF (8) and, thus, the apical dental root portion (6) is in contact with such fluid, whereas the superior chamber (1) receives the culture medium BHI (5) with Enterococcus Faecalis.

All Falcon tubes were left at 37° C. for 30 days. After this period the tubes were opened and serial dilutions were performed. Agar plates were inoculated with 50 μl of each dilution and left in proper gaseous conditions for 24 to 48 h. Results attested the capacity of both, CAC and MTA, to avoid bacterial microleakage and showed 100% of absence of bacterial growth.

### Example 6

Histopathological “in vitro” tests were carried out in order to assess the progress of a primary culture of osteogenic cells from newborn mice calvaria in contact with tiny discs of CAC and MTA. These cementitious discs had standard dimensions such as 5 mm of diameter and 2 mm of height. Before cell culture, discs were gamma rays sterilized and thereafter put on polystyrene plates with 24 wells. Culture itself was made by 20000 cells/well where osteoblasts could undergo differentiation. Such differentiation, whose activity favors the onset of an osteo extracellular matrix mineralization, was evidenced by the alkaline phosphatase measured by the Fast Red histochemical method. The cell structures, monitored by the actin cytoskeleton, as well as the cell density around the discs were evaluated by histochemical techniques using Phalloidin/DAPI. The results showed higher osteogenic cell density near to CAC discs when compared to MTA ones at the end of 7 days.

1-33. (canceled)

34. A composition based on calcium aluminate cement, for use in endodontics, comprising:
(a) calcium aluminate cement comprising oxides in weight percentages: Al₂O₃≧68; CaO≦31; SiO₂=0.3-0.8; MgO=0.4-0.5; and Fe₂O₃<0.3; and
(b) additives in weight percentage relative to cement: (i) dispersant at a content of 0.4 to 0.8; (ii) plasticizer at a content of 2.0 to 4.0; and (iii) radiopaque agent at a content of 20 to 35; and
(c) water to complete the composition, so that water/cement ratio is in the range of 0.19 to 0.24.

35. The composition of claim 34, wherein the dispersant is present at a content of 0.6% by weight of the cement in said composition, the dispersant comprising:
(i) citric acid and/or salts, thereof;
(ii) polyacrylic/polymethacrylic acid and/or salts thereof;
(iii) phosphoric acid and/or salts thereof; and/or
(iv) polyglycol family polymers.

36. The composition of claim 34, wherein the plasticizer is present at a content of 2.8% by weight of the cement, the plasticizer comprising CaO, Ca(OH)₂, CaCO₃, and/or CaCl₂.

37. The composition of claim 34, wherein the radiopaque agent is present at a content of up to 35% by weight of the cement, the radiopaque agent comprising ZrO₂, SnO₂, BaSO₄, Bi₂O₃, Bi₅O(OH)₉(NO₃)₄, CHI₃, and/or ZnO.

38. The composition of claim 34, further comprising a setting retarder at a content of 0.02 to 0.5% by weight of the cement, the setting retarder comprising, citric acid and/or salts thereof.

39. The composition of claim 34, further comprising a setting accelerator at a content of 0.005 to 0.1% by weight of the cement, the accelerator comprising a lithium salt.

40. The composition of claim 34, comprising the preparation steps of:
(a) mixing the cement with the additives, i.e., the dispersant, plasticizer, and radiopaque agent, until a homogeneous blend is achieved;
(b) further homogenizing the blend for 1 hour to 3 hours through a double-cone mill containing 40-80 g of Al₂O₃ mill balls per 100 g of material previously prepared according to step (a), the mill balls being added at a proportion of 60 g per 100 g of material previously prepared according (a).

41. A cementitious product obtained after hardening of a composition comprising:
(a) calcium aluminate cement (CAC) comprising oxides in weight percentage: Al₂O₃≧68; CaO≦31; SiO₂=0.3-0.8; MgO=0.4-0.5; and Fe₂O₃<0.3; the product also having from zero or more of the following additives:
(b) additives in weight percentage relative to cement: (i) dispersant at a content of 0.4 to 0.8; (ii) plasticizer at a content of 2.0 to 4.0; and (iii) radiopaque agent at a content of 20 to 35;
(c) water to complete the composition, so that water/cement ratio is in the range of 0.19 to 0.24;
after a setting time in the range of 1 to 30 days, the product being characterized by 32.0 to 54.0 MPa for neat CAC, 54.4 to 59.6 MPa for CAC with one or more additives other than a radiopaque agent, and 52.0 to 77.7 MPa for CAC with one or more additives, at least one additive being a plus radiopaque agent.

42. The cementitious product of claim 41, further characterized by: (i) providing an ionic conductivity from 0 to 92.60 μS/cm within a period of 0 to 160 minutes of the setting time; and (ii) developing pH of 9 to 12.63 within the same period.

43. The cementitious product of claim 41, further characterized by presenting apparent porosity after the setting time of between 21.74% and 15.89% for neat CAC, between 16.03% and 6.31% for CAC with additives other than a radiopaque agent, and between 9.17% and 2.03% for CAC with one or more additives, at least one being a radiopaque agent.

44. The cementitious product of claim 41, characterized by evidencing higher osteogenic cell density close to CAC when compared to endodontic suitable MTA repair cements at the end of 7 days from setting time.

45. The cementitious product of claim 41, wherein the additives include a dispersant comprising modified polycarboxylic ether and a plasticizer comprising CaCl₂.

46. The cementitious product of claim 41 wherein the composition includes a radiopaque agent comprising Bi₂O₃.

47. A method of using a cementitious product of claim 41:
providing the composition;
applying the composition to a patient in one more the following applications (i) as a repair cement without inducing tooth darkening along the time; (ii) as a repair cement for pulp capping, as well as for root perforations and root canal filling; and (iii) as a pulp cavity basement;
allowing the composition to harden in the applied application.

48. The cementitious product of claim 43 wherein the product is further characterized as having pore diameters or from 2.5 μm down to 0.25 μm.

49. The composition of claim 35 wherein the dispersant comprises a selection from the group of: sodium citrate, sodium polymethylacrylate, sodium hexametaphosphate, and modified polycarboxylic ether.

50. The product of claim 41 wherein the composition includes a dispersant comprising a selection from the group of: sodium citrate, sodium polymethylacrylate, sodium hexametaphosphate, and modified polycarboxylic ether.

51. The composition of claim 37 wherein the radiopaque agent comprises Bi₂O₃.

52. The product of claim 41 wherein the composition includes a radiopaque agent comprising Bi₂O₃.

53. The product of claim 41 wherein the composition includes a plasticizer comprising CaCl₂.
